(19) United States
(12) Patent Application Publication
(10) Pub. No.: US 2026/0263069 A1
(43) Pub. Date: Sep. 10, 2026

(54) STAPLING DEVICE WITH FIRING LOCKOUT ASSEMBLY

(71) Applicant: Covidien LP, Mansfield, MA (US)

(72) Inventors: Pawel ABRAMEK, Berlin, CT (US); Kenneth M. CAPPOLA, Monroe, CT (US); Kenneth H. WHITFIELD, North Haven, CT (US)

(21) Appl. No.: 19/162,428

(22) PCT Filed: Mar. 19, 2024
(86) PCT No.: PCT/US2024/020581

§ 371 Date: Sep. 5, 2025

Related U.S. Application Data
(60) Provisional application No. 63/453,220, filed on Mar. 20, 2023.

(51) Int. Cl. A61B 17/072; A61B 17/00; A61B 90/00

(52) U.S. Cl. CPC A61B 17/07207; A61B 90/03; A61B 90/08; A61B 2017/00477; A61B 2017/07271; A61B 2017/07278; A61B 2017/07285; A61B 2090/034

(57) ABSTRACT

A surgical stapling device includes an elongate body that supports a tool assembly including a cartridge assembly having a replaceable staple cartridge. The tool assembly is supported on the elongate body by a mounting assembly that supports a lockout assembly. The lockout assembly is provided to prevent firing of the stapling device when an actuation sled of the cartridge assembly is not positioned in a proximal portion of the stapling device and when a knife is not supported on the actuation sled.

### FIELD

This technology is generally related to surgical stapling devices, and more particularly, to surgical stapling devices including lockout assemblies.

### BACKGROUND

Surgical stapling devices configured for endoscopic use are commonly used during surgical procedures to minimize patient trauma and reduce patient recovery times. Typically, endoscopic stapling devices include a tool assembly and a drive assembly that is movable in relation to the tool assembly to actuate the tool assembly. The drive assembly includes a knife bar having a cutting blade for cutting tissue. The tool assembly includes anvil and cartridge assemblies that are coupled to each other by a pivot member and movable in relation to each other between unclamped and clamped positions in response to movement of the drive assembly from a retracted position to an advanced position. The cartridge assembly includes a staple cartridge that includes a cartridge body that supports staples and an actuation sled that is movable through the cartridge body in response to movement of the drive assembly from the retracted position to the advanced position to eject the staples from the cartridge body.

Some stapling devices include a staple cartridge that can be replaced after each firing of the stapling device to facilitate reuse of the stapling device. To ensure that a stapling device is not fired with a spent staple cartridge, i.e., a staple cartridge having no staples, or without a staple cartridge, some stapling devices include lockouts.

A continuing need exists for a surgical stapling device having a cost-effective assembly for preventing firing of the stapling device when the stapling device includes a spent staple cartridge or an inoperative staple cartridge.

### SUMMARY

Aspects of this disclosure are directed to a surgical stapling device that includes an elongate body that supports a tool assembly including a cartridge assembly having a replaceable staple cartridge. The tool assembly is supported on the elongate body by a mounting assembly that supports a lockout assembly. The lockout assembly is provided to prevent firing of the stapling device when an actuation sled of the cartridge assembly is not positioned in a proximal portion of the tool assembly and/or when a knife is not supported on the actuation sled within the tool assembly.

Aspects of the disclosure are directed to a surgical stapling device including a mounting assembly, a tool assembly, a drive assembly, and a lockout assembly. The mounting assembly includes a first mounting portion and a second mounting portion that is secured to the first mounting portion to define a channel. The tool assembly is secured to the mounting assembly and includes an anvil and a cartridge assembly. The cartridge assembly includes a staple cartridge and a channel, and the channel has a proximal portion and a distal portion and defines a cavity. The staple cartridge is supported within the cavity of the channel and includes a cartridge body, an actuation sled assembly, staples, and pushers. The cartridge body defines a knife slot and staple receiving pockets positioned on opposite sides of the knife slot, and the staples are received within the staple receiving pockets. The actuation sled assembly is movable through the cartridge body from a sled retracted position to a sled advanced position into engagement with the pushers to eject the staples from the cartridge body. The actuation sled assembly includes an actuation sled having an abutment member. The drive assembly includes a flexible drive beam and a working member and is movable between a drive retracted position and a drive advanced position to move the actuation sled assembly from the sled retracted position to the sled advanced position. The flexible drive beam extends through the longitudinal channel of the mounting assembly and includes a stop surface and a distal portion secured to the working member. The lockout assembly includes a latch and a biasing member. The latch includes a proximal portion and a distal portion and is pivotably secured to the mounting assembly and movable between a locked position and an unlocked position. The proximal portion of the latch includes a stop member, and the distal portion of the latch includes a finger and an actuator. The finger supports the biasing member such that the biasing member is positioned between the mounting assembly and the latch to urge the latch towards the locked position. The actuator is positioned to be engaged by the abutment member of the actuation sled as the actuation sled moves from the sled retracted position towards the sled advanced position to move the latch from the locked position to the unlocked position. In the locked position, the stop member of the latch is aligned with the stop surface of the drive assembly.

In aspects of the disclosure, the actuation sled of the actuation sled assembly includes a central body portion, and the actuation sled assembly includes a knife that is supported on the central body portion of the actuation sled.

In some aspects of the disclosure, the knife is positioned to engage the working member of the drive assembly as the drive assembly moves from the drive retracted position towards the drive advanced position to move the actuation sled assembly from the sled retracted position to the sled advanced position.

In certain aspects of the disclosure, the knife is positioned proximally of the central body portion of the actuation sled.

In aspects of the disclosure, a distance between the working member of the drive assembly and the central body portion of the actuation sled when the drive assembly is in the drive retracted position and the actuation sled assembly is in the sled retracted position is greater than a distance between the stop surface of the drive assembly and the stop member of the latch of the lockout assembly.

In some aspects of the disclosure, the knife is supported on the actuation sled for movement between a lowered position and a raised position.

In certain aspects of the disclosure, the working member of the drive assembly includes a first beam, a second beam, and a vertical strut connecting the first beam to the second beam.

In aspects of the disclosure, the vertical strut defines a cam slot, and the knife includes a cam member that is received in the cam slot when the drive assembly is moved from the drive retracted position towards the drive advanced position to move the knife from the lowered position to the raised position.

In some aspects of the disclosure, the anvil is fixedly secured to the mounting assembly, and the cartridge assembly is pivotably secured to the mounting assembly and movable in relation to the anvil between the unclamped position and the clamped position.

In certain aspects of the disclosure, the latch includes a central portion that includes at least one opening and the mounting assembly includes at least one pivot member that is received within the at least one opening to pivotably couple the latch to the mounting assembly.

In aspects of the disclosure, the proximal portion of the latch is U-shaped and includes two spaced longitudinally extending legs, and the stop member extends transversely between proximal portions of the longitudinally extending legs.

In some aspects of the disclosure, the proximal portion of the latch is L-shaped and includes a longitudinally extending leg, and the stop member extends transversely from a proximal portion of the longitudinally extending leg.

In certain aspects of the disclosure, the actuation sled includes a base having a cutout positioned proximally of the central body portion of the actuation sled, and the cutout is positioned to receive the working member of the drive assembly when the drive assembly is moved from the drive retracted position towards the drive advanced position.

In aspects of the disclosure, the working member has an I-beam configuration and includes a first beam, a second beam, and a vertical strut that interconnects the first beam and the second beam, and the vertical strut includes a distal surface that engages the knife of the actuation sled assembly when the drive assembly is moved from the drive retracted position towards the drive advanced position.

In some aspects of the disclosure, the distal surface of the vertical strut of the drive assembly defines a recess that receives the knife when the drive assembly is moved from the drive retracted position towards the drive advanced position.

Other aspects of the disclosure are directed to a surgical stapling device that includes a mounting assembly, a tool assembly, a drive assembly, and a lockout assembly. The mounting assembly includes a first mounting portion and a second mounting portion secured to the first mounting portion to define a channel. The tool assembly is secured to the mounting assembly and includes an anvil and a cartridge assembly. The cartridge assembly includes a staple cartridge and a channel. The channel has a proximal portion and a distal portion and defines a cavity. The staple cartridge is supported within the cavity of the channel and includes a cartridge body, an actuation sled assembly, staples, and pushers. The cartridge body defines a knife slot and staple receiving pockets positioned on opposite sides of the knife slot, and the staples are received within the staple receiving pockets of the cartridge body. The actuation sled assembly is movable through the cartridge body from a sled retracted position to a sled advanced position into engagement with the pushers to eject the staples from the cartridge body. The actuation sled assembly includes an actuation sled having a central body portion and a knife supported on the central body portion. The knife extends proximally of the central body portion of the actuation sled. The drive assembly includes a stop surface and is positioned to engage the knife of the actuation sled assembly as the drive assembly moves from the drive retracted position towards the drive advanced position to move the actuation sled assembly from the sled retracted position to the sled advanced position. The lockout assembly has a latch that is pivotably secured to the mounting assembly and movable between a locked position and an unlocked position. The latch includes a stop member that is aligned with the stop surface of the drive assembly in the locked position. A distance between the drive assembly and the central body portion of the actuation sled when the drive assembly is in the drive retracted position and the actuation sled assembly is in the sled retracted position is greater than a distance between the stop surface of the drive assembly and the stop member of the latch of the lockout assembly such that the latch will prohibit movement of the drive assembly to the drive advanced position when the knife is not present in the staple cartridge.

In aspects of the disclosure, the knife is supported on the actuation sled for movement between a lowered position and a raised position.

In some aspects of the disclosure, the working member of the drive assembly includes a first beam, a second beam, and a vertical strut that connects the first beam to the second beam.

In certain aspects of the disclosure, the vertical strut defines a cam slot, and the knife includes a cam member that is received in the cam slot when the drive assembly is moved from the drive retracted position towards the drive advanced position to move the knife from the lowered position to the raised position.

In aspects of the disclosure, the lockout assembly includes a biasing member to urge the latch towards the locked position.

In some aspects of the disclosure, the latch includes a finger and an actuator, and the finger supports the biasing member to urge the latch towards the locked position.

In certain aspects of the disclosure, the actuator is positioned to be engaged by the abutment member of the actuation sled as the actuation sled moves from the sled retracted position towards the sled advanced position to move the latch from the locked position to the unlocked position.

In aspects of the disclosure, the latch includes a proximal portion that has a U-shape and includes two spaced longitudinally extending legs, and the stop member extends transversely between proximal portions of the longitudinally extending legs.

Other aspects of the disclosure are directed to a surgical stapling device including a mounting assembly, a tool assembly, a drive assembly, and a lockout assembly. The tool assembly is secured to the mounting assembly and includes an anvil and a cartridge assembly. The cartridge assembly includes a staple cartridge and a channel, and the channel has a proximal portion and a distal portion and defines a cavity. The staple cartridge is supported within the cavity of the channel and includes a cartridge body, an actuation sled, staples, and pushers. The cartridge body defines a knife slot and staple receiving pockets positioned on opposite sides of the knife slot, and the staples are received within the staple receiving pocket. The actuation sled is movable through the cartridge body from a sled retracted position to a sled advanced position into engagement with the pushers to eject the staples from the cartridge body. The drive assembly includes a stop surface and is movable between a drive retracted position and a drive advanced position to move the actuation sled assembly from the sled retracted position to the sled advanced position. The lockout assembly has a latch that is pivotably secured to the mounting assembly and is movable between a locked position and an unlocked position. The latch includes a proximal portion and a distal portion. The proximal portion of the latch includes a stop member, and the distal portion of the latch includes an actuator that is positioned to be engaged by the abutment member of the actuation sled as the actuation sled moves from the sled retracted position towards the sled advanced position to move the latch from the locked position to the unlocked position. In the locked position, the stop member of the latch is aligned with the stop surface of the drive assembly.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of a surgical stapling device according to aspects of the disclosure with a tool assembly of the stapling device in an unclamped position, pre-fired position;

FIG. 2 is side perspective view of the tool assembly of the stapling device shown in FIG. 1 with a staple cartridge of a cartridge assembly of the surgical stapling device separated from a channel of the cartridge assembly;

FIG. 3 is an exploded perspective view of the staple cartridge of the tool assembly shown in FIG. 2;

FIG. 4 is a side perspective view of an actuation sled assembly of the staple cartridge shown in FIG. 3;

FIG. 5 is a perspective view from the proximal end of the actuation sled assembly shown in FIG. 3;

FIG. 6 is an exploded view of the actuation sled assembly shown in FIG. 5;

FIG. 7 is an exploded view of the tool assembly, mounting assembly, lockout assembly, and drive assembly of the stapling device shown in FIG. 1 with the staple cartridge of the tool assembly removed;

FIG. 8 is an enlarged exploded view of the indicated area of detail shown in FIG. 7 illustrating a portion of the mounting assembly and the lockout of the stapling device shown in FIG. 1;

FIG. 9 is a side perspective view of the portion of the mounting assembly and the lockout shown in FIG. 8 assembled;

FIG. 10 is a cross-sectional view taken along section line 10-10 of FIG. 2 illustrating a proximal portion of the tool assembly, the mounting assembly and the lockout assembly of the stapling device shown in FIG. 1 prior to installation of the staple cartridge into the channel of the cartridge assembly;

FIG. 11 is an enlarged view of the indicated area of detail shown in FIG. 7 illustrating a distal portion of the drive assembly;

FIG. 12 is a perspective view from the proximal end of the staple cartridge shown in FIG. 2;

FIG. 13 is a perspective view from above of the tool assembly of the stapling device shown in FIG. 1 in the unclamped, pre-fired position with an anvil of the tool assembly shown in phantom;

FIG. 14 is an enlarged view of the indicated area of detail shown in FIG. 13;

FIG. 15 is a cross-sectional view taken through the tool assembly and a distal portion of an elongate body of the stapling device shown in FIG. 1 with the tool assembly of the stapling device in an unclamped, pre-fired position;

FIG. 16 is an enlarged view of the indicated area of detail shown in FIG. 15;

FIG. 17 is a cross-sectional view taken along a centerline of a proximal portion of the tool assembly and a distal portion of the elongate body of the stapling device shown in FIG. 1 with the tool assembly of the stapling device in an unclamped, pre-fired position;

FIG. 18 is a cross-sectional view taken along the centerline of the proximal portion of the tool assembly and the distal portion of the elongate body of the stapling device shown in FIG. 1 with the tool assembly of the stapling device in a clamped, pre-fired position;

FIG. 19 is a cross-sectional view taken along an axis offset from the centerline of the proximal portion of the tool assembly and the distal portion of the elongate body of the stapling device shown in FIG. 1 with the tool assembly of the stapling device in a clamped, pre-fired position;

FIG. 20 is a cross-sectional view taken along the centerline of the proximal portion of the tool assembly and the distal portion of the elongate body of the stapling device shown in FIG. 1 with the tool assembly of the stapling device in a clamped position as the surgical stapling device begins to be fired;

FIG. 21 is a cross-sectional view taken along an axis offset from the centerline of the proximal portion of the tool assembly and the distal portion of the elongate body of the stapling device shown in FIG. 1 with the tool assembly of the stapling device in a clamped position as the drive assembly begins to advance to fire the surgical stapling device;

FIG. 22 is a cross-sectional view taken along an axis offset from the centerline of the proximal portion of the tool assembly and the distal portion of the elongate body of the stapling device shown in FIG. 1 with the tool assembly of the stapling device in a clamped position as the surgical stapling device as the drive assembly begins to advance to fire the surgical stapling device;

FIG. 23 is a cross-sectional view taken along an axis offset from the centerline of the proximal portion of the tool assembly and the distal portion of the elongate body of the stapling device shown in FIG. 1 with the tool assembly of the stapling device in a clamped, and fired position and the drive assembly in the advanced position;

FIG. 24 is a side perspective view of the actuation sled of the actuation sled assembly shown in FIG. 4 with the knife of the actuation sled assembly missing;

FIG. 25 is a cross-sectional view taken through a proximal portion of the tool assembly and a distal portion of the elongate body of the surgical stapling device shown in FIG. 1 with the knife missing from the actuation sled assembly in the clamped, pre-fired position;

FIG. 26 is an exploded view of an alternate version of a portion of the mounting assembly and the lockout assembly shown in FIG. 8.

FIG. 27 is a perspective view from the distal end of the portion of the mounting assembly and the lockout assembly shown in FIG. 26; and

FIG. 28 is a perspective view from above of the portion of the mounting assembly and the lockout assembly shown in FIG. 26.

### DETAILED DESCRIPTION

The disclosed surgical stapling device will now be described in detail with reference to the drawings in which like reference numerals designate identical or corresponding elements in each of the several views. However, it is to be understood that disclosed aspects of the surgical stapling device are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

The disclosure is directed to a surgical stapling device that includes an elongate body that supports a tool assembly including a cartridge assembly having a replaceable staple cartridge. The tool assembly is supported on the elongate body by a mounting assembly that supports a lockout assembly. The lockout assembly is provided to prevent firing of the stapling device when an actuation sled of the cartridge assembly is not positioned in a proximal portion of the stapling device and/or when a knife is not supported on the actuation sled.

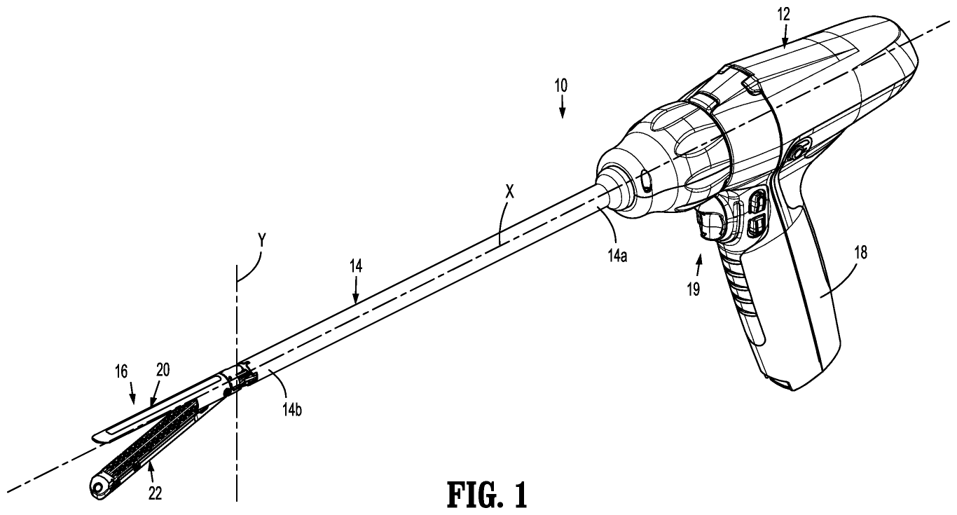

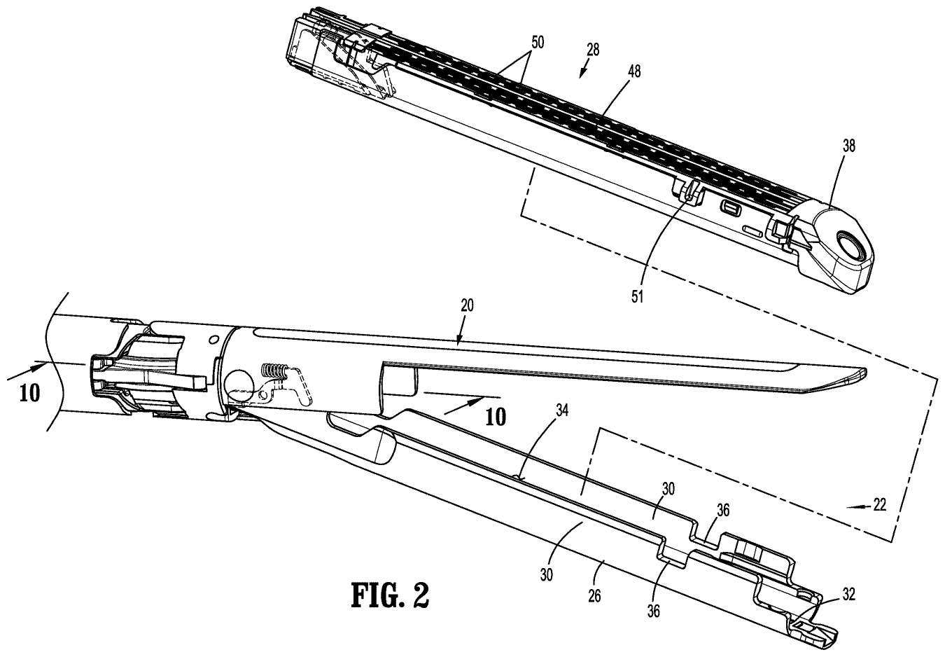

FIGS. 1 and 2 illustrate a surgical stapling device shown generally as stapling device 10 that includes a handle assembly 12, an adapter assembly 14, and a tool assembly 16. The handle assembly 12 is powered and includes a stationary handgrip 18 and an actuation button or buttons 19. The actuation buttons 19 are operable to actuate various functions of the tool assembly 16 via the adapter assembly 14, i.e., approximation of the tool assembly 16, and stapling and cutting of tissue. In certain aspects of the disclosure, the handle assembly 12 supports batteries (not shown) that provide power to the handle assembly 12 to operate the stapling device 10. Although the stapling device 10 is illustrated as a powered stapling device, it is envisioned that the advantages of this disclosure are suitable for use with manually powered surgical stapling devices as well as robotically controlled stapling devices. U.S. Pat. No. 5,865,361 describes a stapling device that includes exemplary aspects of a manually powered stapling device.

The adapter assembly 14 defines a longitudinal axis “X” and includes a proximal portion 14a and a distal portion 14b. The proximal portion 14a of the adapter assembly 14 is coupled to the handle assembly 12, and the distal portion 14b of the adapter assembly 14 supports the tool assembly 16. In aspects of the disclosure, the tool assembly 16 is pivotably coupled to the distal portion 14b of the adapter assembly to facilitate articulation of the tool assembly 16 in relation to the adapter assembly 14 about an articulation axis “Y”.

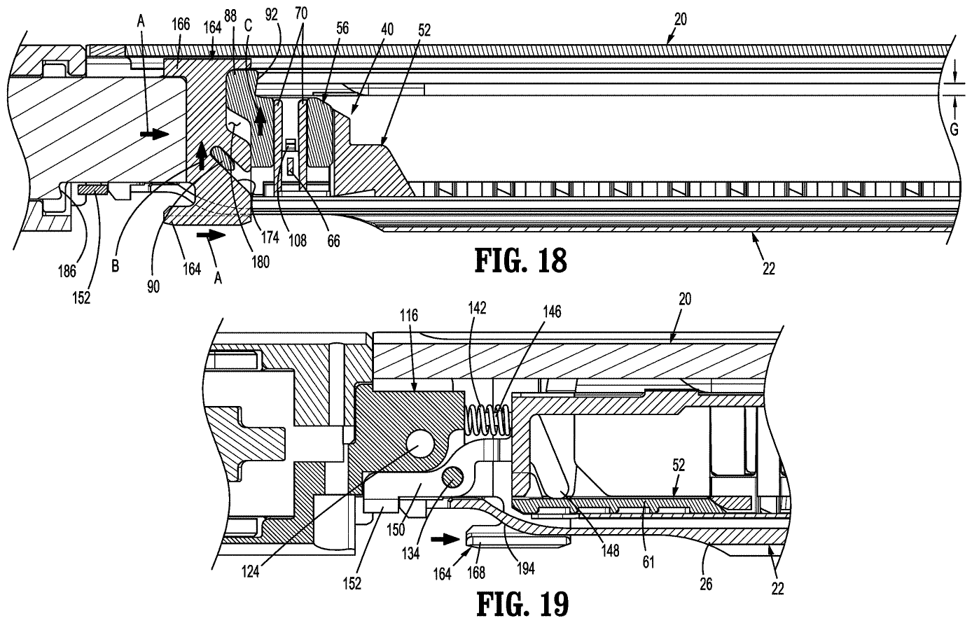

The tool assembly 16 includes an anvil 20 and a cartridge assembly 22. In aspects of the disclosure, the cartridge assembly 22 is pivotably supported in relation to the anvil 20 to facilitate movement of the tool assembly 16 between an unclamped position (FIG. 1) and a clamped position (FIG. 18).

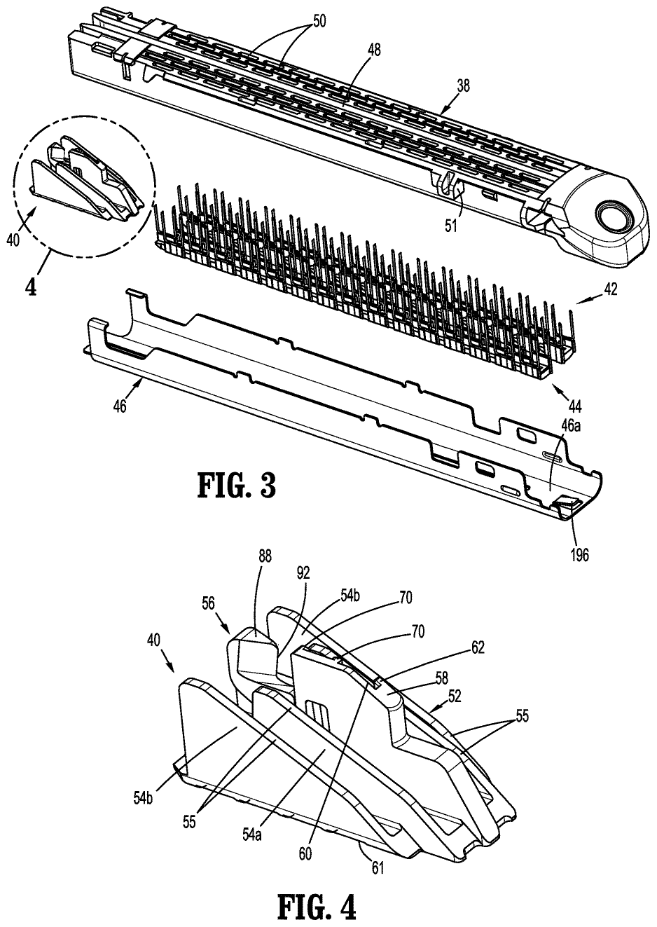

FIGS. 2 and 3 illustrate the cartridge assembly 22 which includes a channel 26 and a staple cartridge 28. The channel 26 includes side walls 30 and a bottom wall 32 that define a cavity 34. The staple cartridge 28 is removably received within the cavity 34 of the channel 26 and is replaceable to facilitate reuse of the stapling device 10. Each of the side walls 30 of the channel 26 has an upper end that defines a recess 36.

The staple cartridge 28 includes a cartridge body 38, an actuation sled assembly 40, staples 42, pushers 44, and a staple guard 46. The cartridge body 38 defines a knife slot 48 and staple receiving pockets 50 that are positioned on each side of the knife slot 48. In aspects of the disclosure, the staple receiving pockets 50 are aligned in two or more rows on opposite sides of the knife slot 48. In other aspects of the disclosure, the knife slot 48 is centrally located in the cartridge body 38. However, it is envisioned that the knife slot 48 could be offset from the central axis of the cartridge body 38.

The cartridge body 38 includes laterally extending protrusions 51 (only one is shown) that are received within the recesses 36 (FIG. 2) of the channel 26 to properly position the staple cartridge 28 within the cavity 34 of the channel 26. The staples 42 and the pushers 44 are received within the staple receiving pockets 50 of the cartridge body 38, and the staple guard 46 is secured to the bottom of the cartridge body 38 to retain the staples 42 and pushers 44 within the cartridge body 38. The actuation sled assembly 40 is movable through the cartridge body 38 from a retracted position to an advanced position and includes an actuation sled 52 having angled inner and outer cam members 54a, 54b (FIG. 4). The cam members 54a, 54b have angled cam surfaces 55 that are positioned to sequentially engage the pushers 44 as the actuation sled 52 moves through the cartridge body 38 to lift the pushers 44 within the staple receiving pockets 50 and eject the staples 42 from the cartridge body 38.

FIGS. 4-6 illustrate the actuation sled assembly 40 which includes the actuation sled 52 and a knife 56. The actuation sled 52 includes the cam members 54a, 54b, a central body portion 58, and abutment members or fins 59. In aspects of the disclosure, the fins 59 are positioned proximally and laterally outwardly of cam members 54a, 54b. The cam members 54a, 54b are positioned on opposite sides of the central body portion 58, and each of the fins 59 is longitudinally aligned and positioned proximally of one of the inner cam members 54a.

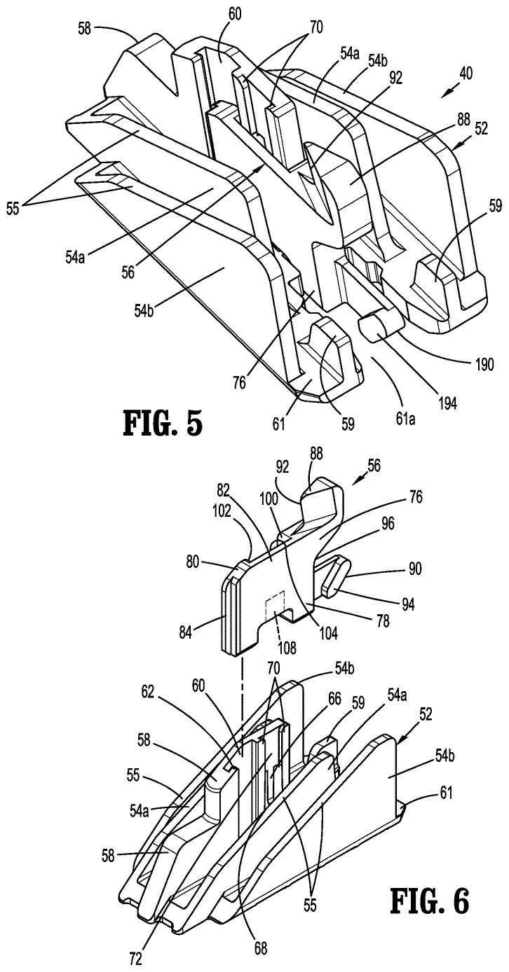

The actuation sled 52 includes a base portion 61 that is supported on a bottom wall 46a (FIG. 3) of the staple guard 46 and defines a proximal cutout 61a (FIG. 5). The central body portion 58 and the cam members 54a, 54b extend upwardly from the base portion 61 of the actuation sled 52, with the central body portion positioned between the inner cam members 54a. The central body portion 58 is received within the knife slot 48 of the cartridge body 38 as the actuation sled assembly 40 moves within the cartridge body 38 from the retracted position towards the advanced position.

The central body portion 58 of the actuation sled 52 defines a guide channel 60 (FIG. 6) and includes a lip 62 that extends partially over the guide channel 60. The central body portion 58 also includes a resilient arm 66 that supports an abutment 68. In aspects of the disclosure, the resilient arm 66 is integrally formed with the central body portion 58 and is supported on the central body portion 58 in cantilevered fashion to facilitate inward and outward movement of the abutment 68. It is envisioned that the abutment 68 could be movably supported on the central body portion 58 in a variety of manners. In aspects of the disclosure, the central body portion 58 includes spaced vertical walls 70 that are positioned proximally of the guide channel 60 and are spaced from each other to define a recess 72. The resilient arm 66 is received within the recess 72 and is movable between a first position extending from the recess 72 and a second position within the recess 72.

The knife 56 includes a body 76 having a proximal portion 78, a distal portion 80, and a central portion 82. The distal portion 80 of the knife 56 is received within the guide channel 60 of the actuation sled 52 and includes a distal flange 84 that is engaged by the lip 62 of the of the central body portion 58 of the actuation sled 52 to retain the distal portion 80 of the knife 56 within the guide channel 60. The distal portion 80 of the knife 56 is slidably received within the guide channel 60 of the actuation sled 52 to facilitate vertical movement of the knife 56 in relation to the actuation sled 52 between lowered and raised positions.

The proximal portion 78 of the knife 56 includes a cutting blade 88 and a cam member 90. The cutting blade 88 forms the upper section of the proximal portion 78 of the knife 56 and includes a distally facing cutting edge 92. The cam member 90 is positioned on a lower section of the proximal portion 78 of the knife 56 and includes an elongate, angled protrusion 94. Alternately, the protrusion 94 can have a variety of different geometries including a circular geometry. The proximal portion 78 of the knife 56 defines a cavity 96 that is positioned between the cutting blade 88 and the cam member 90. When the knife 56 is supported on the actuation sled 52, the proximal portion of the knife 56 including the cam member 90 is positioned above the cutout 61a in the base 61 of the actuation sled 52.

The central portion 82 of the knife 56 extends between a proximal wall 100 (FIG. 6) of the distal portion 80 of the knife 56 and a distal wall 102 of the proximal portion 78 of the knife 56. The proximal wall 100, the distal wall 102, and the central portion 82 define a cavity 104 that receives the spaced vertical walls 70 of the actuation sled 52 when the distal portion 80 of the knife 56 is received in the guide channel 60 of the actuation sled 52. When the spaced vertical walls 70 of the actuation sled 52 are received within the cavity 104 of the knife 56, the distal wall 102 of the proximal portion 78 of the knife 56 is in sliding engagement with a distal surface of one of the vertical walls 70 of the actuation sled 52 and the proximal wall 100 (FIG. 6) of the distal portion 80 of the knife 56 is in sliding engagement with a proximal surface of the other of the vertical walls 70 of the actuation sled 52. The central portion 82 of the knife 56 includes a detent 108 that projects into the cavity 104 of the knife 56.

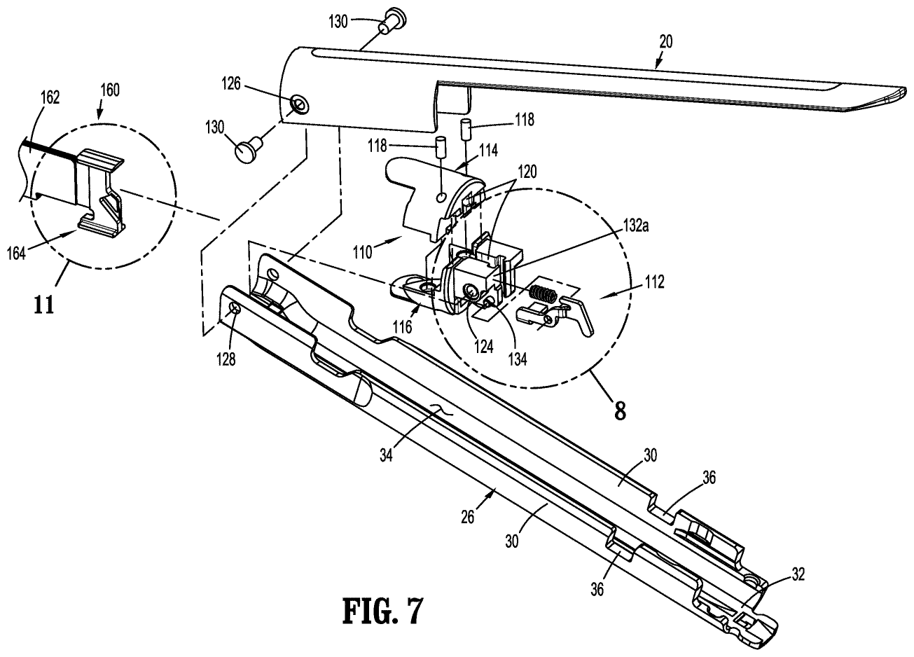

FIG. 7-10 illustrate the tool assembly 16, a mounting assembly 110 that is secured to the proximal portion of the tool assembly 16, and a lockout assembly 112. The mounting assembly 110 includes a first mounting portion 114 and a second mounting portion 116. Although not shown, the first and second mounting portions 114, 116 are pivotably coupled to the distal portion 14b (FIG. 1) of the adapter assembly 14 of the stapling device 10 to facilitate articulation of the tool assembly 16 in relation to the adapter assembly 14 about the articulation axis “Y” (FIG. 1). The first mounting portion 114 is secured to the second mounting portion 116 by pins or rivets 118 to define a longitudinal channel 120 with the second mounting portion 116.

The second mounting portion 116 of the mounting assembly 110 includes a distal portion 116a that is received within the proximal portion of the cavity 34 defined by the channel 26 of the cartridge assembly 22. In aspects of the disclosure, the second mounting portion 116 defines openings 124, and the anvil 20 and the channel 26 of the cartridge assembly 22 define openings 126, 128, respectively. The openings 124, 126, and 128 receive pivot members, e.g., screws or pins 130, to pivotably secure the channel 26 to the anvil 20, and to secure the anvil 20 to the mounting assembly 110. The second mounting portion 110 has a substantially planar distal surface 132a and a lower surface 132b and includes a transversely extending pivot member 134.

The lockout assembly 112 includes a latch 140 and a biasing member 142. The latch 140 defines a centrally located opening 144 that receives the pivot member 134 of the second mounting portion 116 of the mounting assembly 110 to pivotally secure the latch 140 to the mounting assembly 110. The latch 140 is rotatable about the pivot member 134 between a locked position and an unlocked position as described in further detail below. The latch 140 includes a distal portion 144a that is positioned distally of the opening 144 and a proximal portion 144b that is positioned proximally of the opening 144. The distal portion 144a of the latch 140 includes a proximally extending finger 146 and a downwardly extending actuator 148. The biasing member 142, e.g., a coil spring, is received about finger 146 and is engaged with the distal surface 132a of the second mounting portion 116 to urge the latch 140 to the locked position. The actuator 148 is positioned within the proximal portion of the channel 26 to engage the actuation sled 52 of the actuation sled assembly 40 and affect movement of the latch 140 from the locked position to the unlocked position.

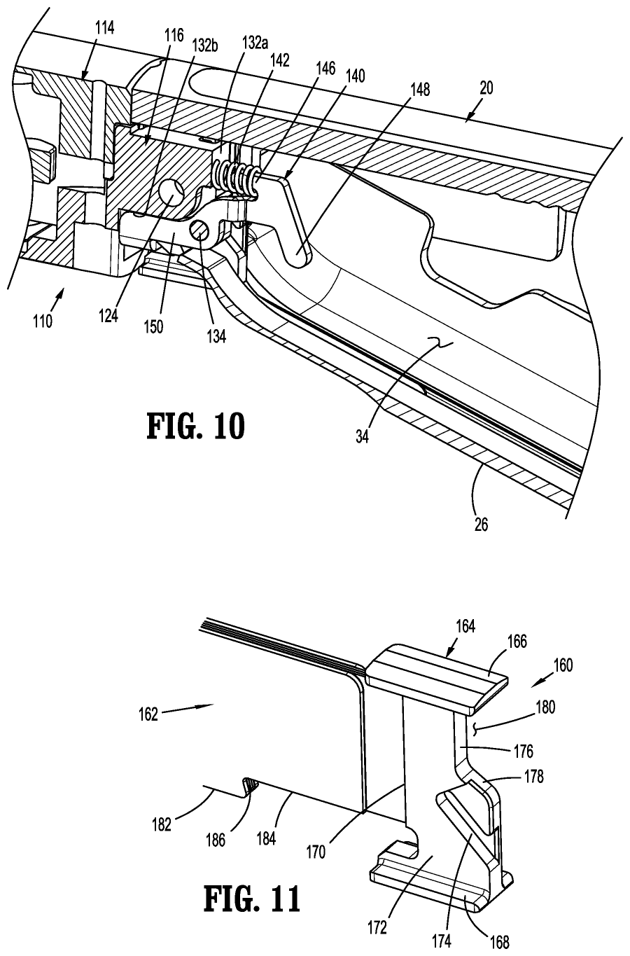

The proximal portion 144b of the latch 140 includes a leg 150 that includes a stop member 152. In aspects of the disclosure, the leg 150 extends proximally from the opening 144 and the stop member 152 extends transversely from a proximal end of the leg 150 across a proximal portion of the longitudinal channel 120 when the latch 140 is in the locked position. The leg 150 is engaged with the lower surface 132b of the second mounting portion 116 when the latch 140 is in the locked position (FIG. 10). Prior to insertion of the staple cartridge 28 into the cavity 34 of the channel 26, the latch 140 is positioned in the locked position with the actuator 148 extending into a proximal portion of the cavity 34 of the channel 26.

FIG. 11 illustrates a distal portion of a drive assembly 160 of the stapling device 10 (FIG. 1) which includes a flexible drive beam 162 and a working member 164. The flexible drive beam 162 includes stacked sheets of material that have distal ends secured to the working member 164. The working member 164 has an I-beam configuration and includes a first beam 166, a second beam 168, and a vertical strut 170 that secures the first and second beams 166, 168 together at a fixed distance from each other. The vertical strut 170 has a side wall 172 that defines a cam slot 174 and a distal surface 176 that includes an angled portion 178 that is angled upwardly and proximally at a position between the cam slot 174 and the first beam 166 to define a recess 180 between the first beam 166 and the angled portion 178. The drive assembly 160 is movable through the cartridge body 38 (FIG. 2) from a retracted position to an advanced position to advance the actuation sled assembly 40 (FIG. 4) through the cartridge body 38 from its retracted position to its advanced position. As the drive assembly 160 moves through the cartridge body 38, the vertical strut 170 of the working member 164 moves through the knife slot 48 (FIG. 2) of the cartridge body 38, and the first and second beams 166, 168 of the working member 164 engage the anvil 20 and the cartridge assembly 22, respectively, to move the tool assembly 16 from the unclamped position to the clamped position and define a maximum tissue gap between the anvil 20 and the cartridge assembly 22 during firing.

The flexible drive beam 162 of the drive assembly 160 is movable through the longitudinal channel 120 formed in the mounting assembly 110 as the drive assembly 160 moves between the retracted and advanced positions and includes a lower surface 182 that defines a cutout 184. The cutout 184 is defined in part by a stop surface 186 that is positioned at the proximal end of the cutout 184. When the latch 140 is in the locked position, the stop member 152 of the latch 140 is longitudinally aligned with the stop surface 186 of the drive assembly 160 to prohibit further movement of the drive assembly 160 towards the advanced position. When the latch 140 is moved to the unlocked position, the stop member 152 is moved downwardly out of alignment with the stop surface 186 of the drive assembly 160 to allow for advancement of the drive assembly 160.

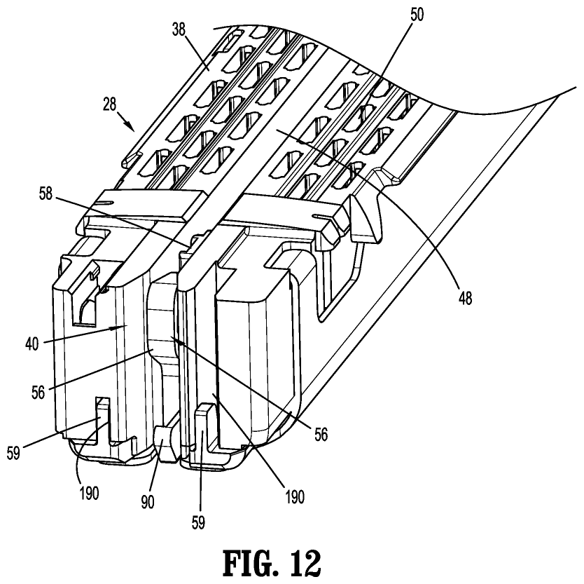

FIG. 12 illustrates the proximal portion of the staple cartridge 28 with the actuation sled assembly 40 in the retracted position within the cartridge body 38. As illustrated, the cam member 90 of the knife 56 is positioned in a proximal end of the knife slot 48 of the cartridge body 38 of the staple cartridge 42. In this position, the abutment 68 (FIG. 6) on the resilient arm 66 of the actuation sled 52 is engaged with the detent 108 on the central portion 82 of the knife 56 to retain the knife 56 in the lowered position, the central body portion 58 of the actuation sled 52 is received in the knife slot 48 of the cartridge body 38, and the fins 59 are received in channels 190 of the cartridge body 38.

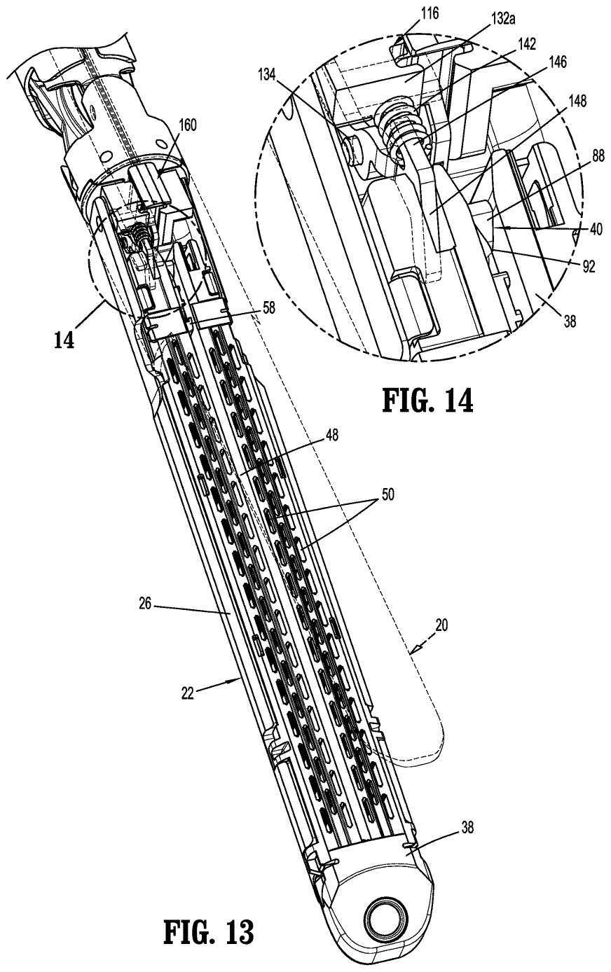

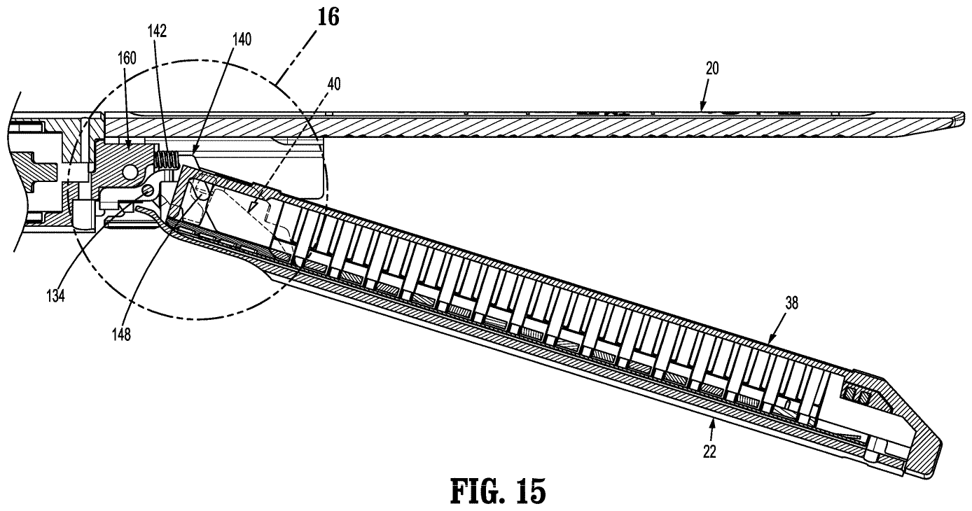

FIGS. 13-17 illustrate the tool assembly 16 in the unclamped, pre-fired position with the staple cartridge 42 received within the channel 26 of the cartridge assembly 22. When the tool assembly 16 is in the unclamped position, the cartridge assembly 22 is pivoted away from the anvil 20 and the actuation sled assembly 40 is in its retracted position in the proximal portion of the cartridge body 38 of the cartridge assembly 22. The drive assembly 160 is also positioned in its retracted position spaced proximally of the cartridge assembly 22, and the first beam 164 of the drive assembly 160 is partially received within a channel 192 defined within the anvil 20. The cam member 90 of the knife 56 is positioned at the proximal end of the cartridge assembly 22 adjacent to the cam slot 174 of the working member 164 of the drive assembly 160, and the knife 56 is in the lowered position (FIG. 17).

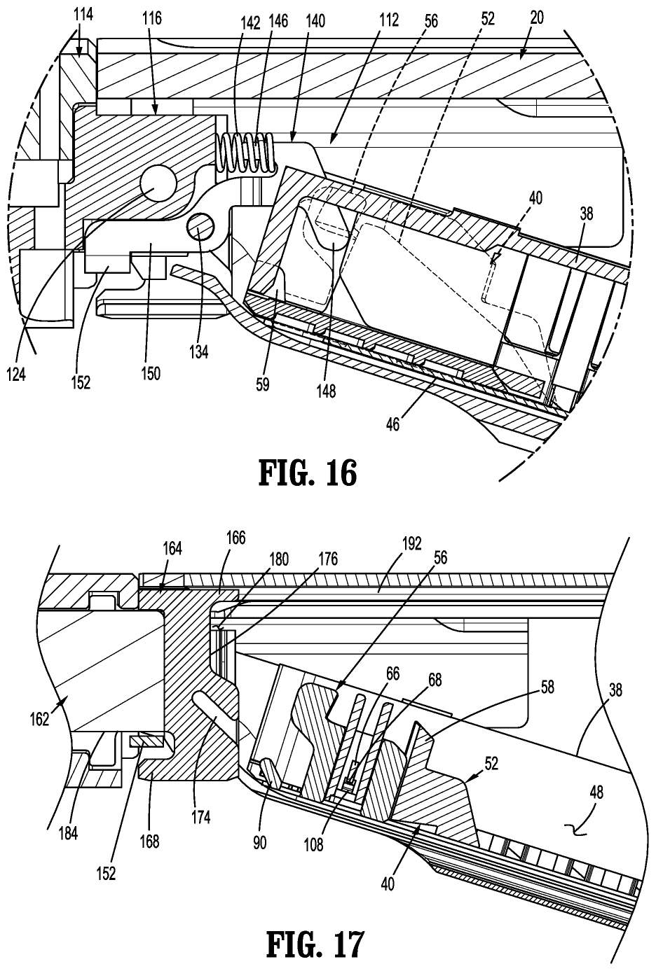

In the unclamped, pre-fired position of the tool assembly 16 (FIG. 16), the latch 140 of the lockout assembly 112 is urged to the locked position by the biasing member 142 to position the stop member 152 of the latch 140 within the cutout 184 of the flexible drive beam 162 of the drive assembly 160 at a position distal of the stop surface 186. The leg 150 of the latch 140 is engaged with the lower surface 132b of the second mounting portion 116 to retain the latch 140 in the locked position.

FIGS. 18 and 19 illustrate the tool assembly 16 of the stapling device 10 (FIG. 1) as the tool assembly 16 is moved to the clamped position. When the drive assembly 160 is moved in the direction indicated by arrows “A” in FIG. 18 from its retracted position towards its advanced position to a clamped position, the second beam 168 of the working member 164 of the drive assembly 160 engages a bend 194 (FIG. 19) in the channel 26 of the cartridge assembly 22 and pivots the cartridge assembly 22 in relation to the anvil 20 to the clamped position, and the working member 164 moves into the cutout 61a (FIG. 5) defined in the base portion 61 of the actuation sled 52. As the cartridge assembly 22 pivots in relation to the anvil 20 towards the clamped position, the cam member 90 of the knife 56 is received in the cam slot 174 of the working member 164 of the drive assembly 160 and is cammed upwardly in the cam slot 174 in the direction indicated by arrow “B” to lift the knife 56 from the lowered position to the raised position in the direction indicated by arrow “C”. In the raised position of the knife 56, the cutting edge 92 of the knife 56 extends from the knife slot 48 of the cartridge body 38 across a tissue gap “G” (FIG. 18) defined between the anvil 20 and the cartridge assembly 22, and the proximal portion of the cutting blade 88 of the knife 52 is received within the recess 180 of the working member 164 of the drive assembly 160. As the knife 56 moves from the lowered position to the raised position, the detent 108 on the central portion 82 of the knife 56 engages the abutment 68 on the resilient arm 66 of the actuation sled 52 and forces the abutment 68 outwardly to allow the knife 56 to move upwardly past the abutment 68 to the raised position. In the clamped position, the latch 140 of the lockout assembly 112 remains in the locked position, but the actuator 148 of the lockout out assembly 112 is positioned on the base portion 61 of the actuation sled 52 of the actuation sled assembly 40.

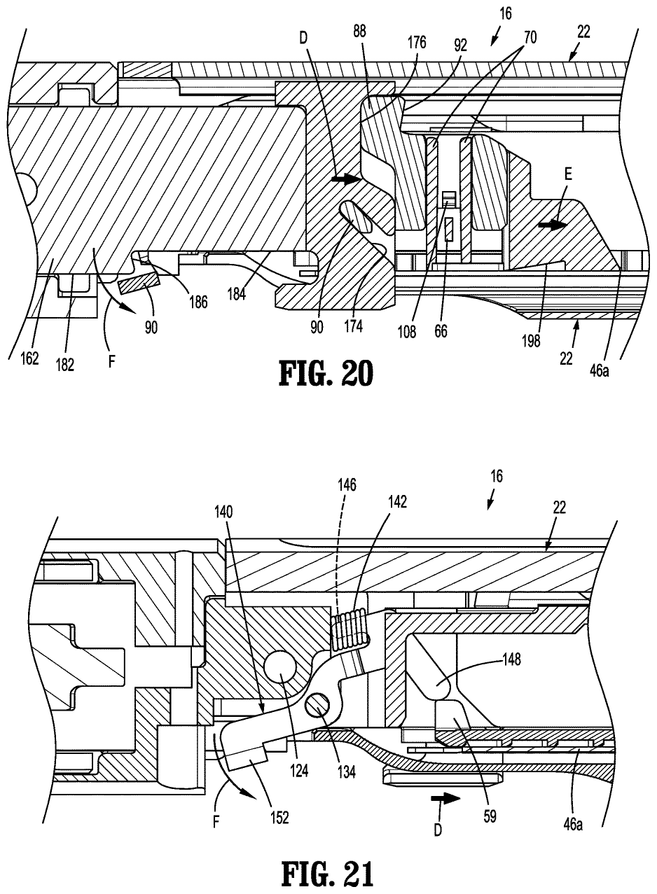

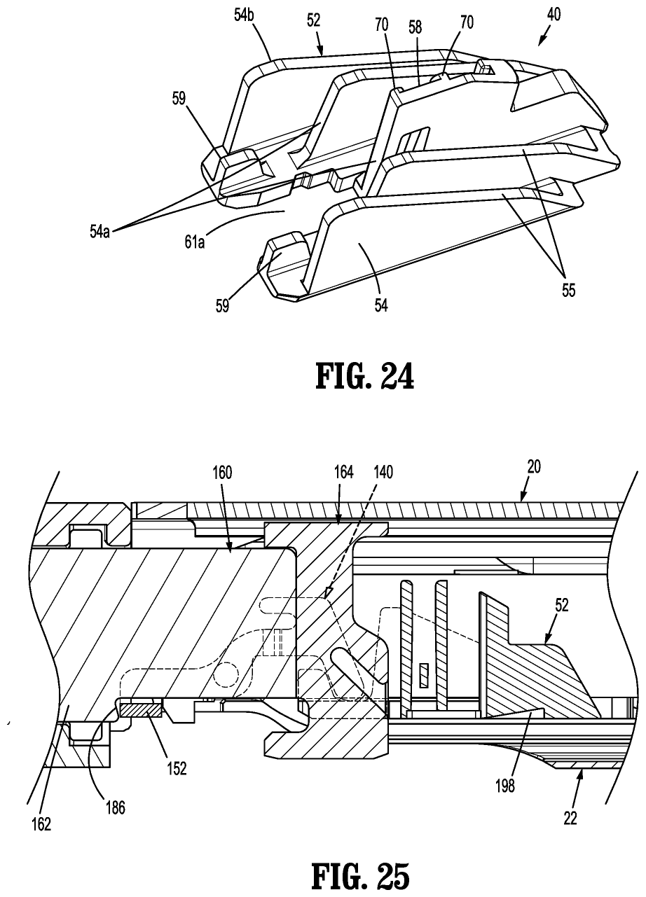

FIGS. 20-23 illustrate the tool assembly 16 of the stapling device 10 (FIG. 1) as the drive assembly 160 moves in the direction indicated by arrows “D” from its clamped position (FIG. 18) towards the advanced position to fire the tool assembly 16. As the drive assembly 160 moves towards its advanced position, the distal surface 176 of the working member 164 of the drive assembly 160, which is in abutting relation with the knife 56, advances the actuation sled assembly 40 in the direction indicated by arrow “E” in FIG. 20 towards its advanced position to eject the staples 42 from the cartridge body 38 and cut tissue clamped between the anvil 20 and the cartridge assembly 22. In aspects of the disclosure, the bottom wall 46a of the staple guard 46 includes a resilient tab 196 (FIG. 23) that extends upwardly from the bottom wall 46a into the path of the actuation sled assembly 40. The resilient tab 196 is supported at its proximal end on the bottom wall 46a in cantilevered fashion. When the actuation sled 52 passes over the resilient tab 196, the resilient tab 196 is deformed downwardly and subsequently snaps into a recess 198 (FIG. 24) formed on the bottom of the actuation sled 52. When the tab 196 is received in the recess 198 of the actuation sled 52, the actuation sled assembly 40 is prevented from moving proximally within the cartridge body 38 of the cartridge assembly 22.

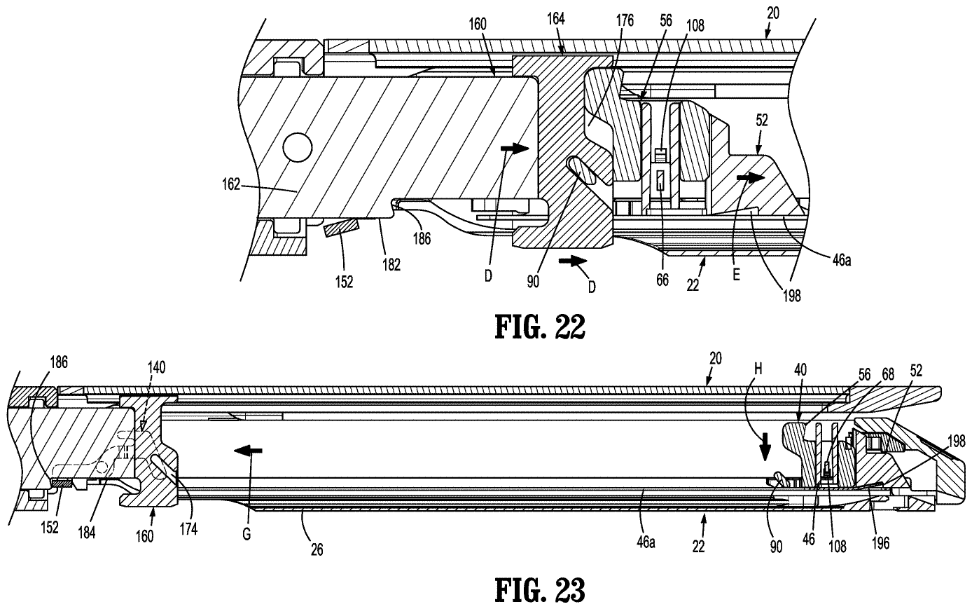

As the drive assembly 160 moves from its clamped position (FIG. 19) towards the advanced position, the fin 59 on the actuation sled 52 engages the actuator 148 of the latch 140 to pivot the latch 140 against the urging of the biasing member 142 in the direction indicated by arrow “F” in FIG. 21 to move the latch 140 to the unlocked position. In the unlocked position, the stop member 152 of the latch 140 is moved to a position below, and out of alignment with the stop surface 186 of the drive assembly 160. In this position, the drive assembly 160 can move past the latch 140 towards its advanced position to advance the actuation sled assembly 40 through the staple cartridge 42 to sequentially eject the staples 42 from the cartridge body 38 of the cartridge assembly 42 and cut tissue clamped between the staple cartridge 42 and the anvil 20. When the fin 59 passes by the actuator 148 of the latch 140, the latch 140 is retained in the unlocked position by engagement of the stop member 152 with the lower surface 182 of the flexible drive beam 162 (FIG. 22). The latch 140 will remain in the unlocked position until the drive assembly 160 is returned to a position near the retracted position in which the stop member 152 is received in the cutout 184 of the flexible drive beam 162 of the drive assembly 160.

FIG. 23 illustrates the tool assembly 16 of the stapling device 10 (FIG. 1) as the drive assembly 160 is moved in the direction indicated by arrow “G” from the advanced position to the retracted position after the stapling device 10 is fired. When the drive assembly 160 is retracted after firing, the actuation sled assembly 40 is retained in its advanced position by the tab 196 on the staple guard 46. As such, the drive assembly 160 moves independently of the actuation sled 52 from the advanced position towards the retracted position to move the cam slot 174 of the working member 164 of the drive assembly 160 in relation to the cam member 90 of the knife 56. This movement cams the knife 56 downwardly in the direction indicated by arrows “H” in FIG. 23 to move the knife 56 from the raised position back to the lowered position and disengage the working member 164 of the drive assembly 160 from the actuation sled assembly 40. As described above, the knife 56 is retained in the lowered position by the engagement of the detent 108 on the central portion 82 of the knife 56 with the abutment 68 on the resilient arm 66 of the actuation sled 52.

In aspects of the disclosure, the pushers 44 include a sharp distal edge sled 52 that is positioned to engage the proximal portion of the actuation sled 52 when the actuation sled 52 is positioned between the sled retracted position and the sled advanced position to prohibit proximal movement of the actuation sled 52 within the cartridge body 38 when the stapling device 10 is in a partially fired position.

When the drive assembly 160 moves to the retracted position, the stop member 152 of the latch 140 is received within the cutout 184 of the flexible drive beam 162 of the drive assembly 160 and the latch 140 is moved to the locked position by the biasing member 142. Since the actuation sled assembly 40 remains in the distal portion of the cartridge body 38 of the cartridge assembly 42, further advancement of the drive assembly is prohibited by the stop member 152 of the latch 140 of the lockout assembly 112. It is noted that if a defective staple cartridge 42 that does not include an actuation sled assembly 40, the lockout assembly 112 will remain in the locked position when the drive assembly 160 is advanced to prohibit firing of the stapling device 10 (FIG. 1).

FIGS. 24 and 25 illustrate the tool assembly 16 of the stapling device 10 in a clamped position when, due to a manufacturing defect, the actuation sled assembly 40 is missing the knife 56 (FIG. 6). When the knife 56 is missing from the actuation sled assembly 40, the working member 164 of the drive assembly 160 is spaced from the proximal portion of the actuation sled 52 when the tool assembly 16 is in the clamped position. More specifically, the vertical strut 170 of the working member 164 is spaced from the central body portion 58 of the actuation sled 52 a distance that is greater than a distance between the stop member 152 of the latch 140 and stop surface 186 of the flexible drive beam 162 of the drive assembly 160. The spacing is dimensioned to allow the stop member 152 of the latch 140 of the lockout assembly 112 to engage the stop surface 186 of the flexible beam 162 before the working member 164 of the drive assembly 160 engages the proximal portion of the actuation sled 52. Thus, the drive assembly 160 will be prohibited by the stop member 152 of the latch 140 from moving to the advanced position when a knife 56 is not present in the cartridge assembly 22.

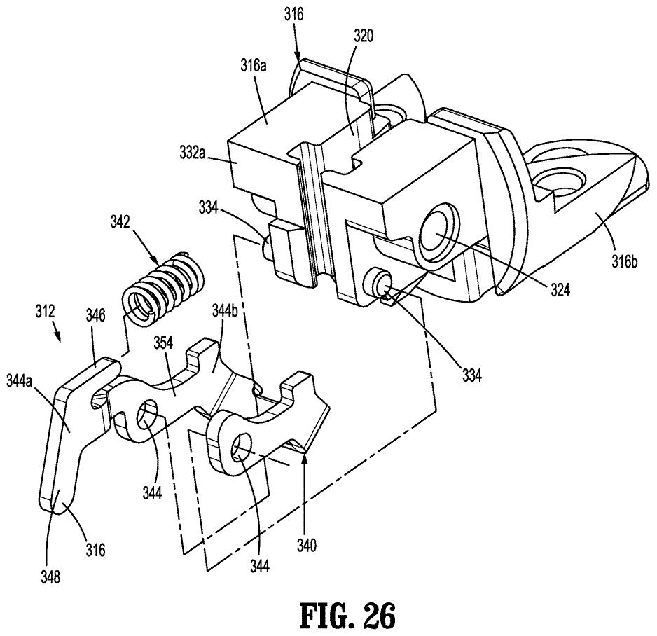

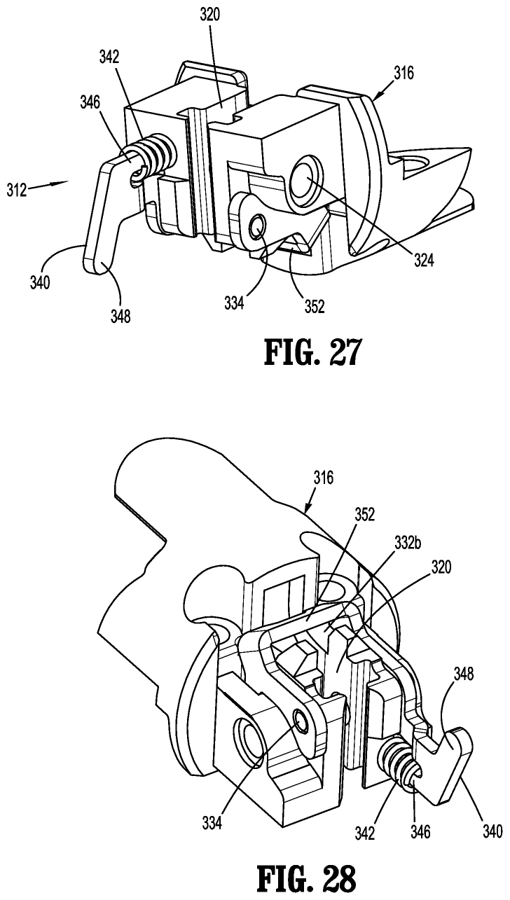

FIGS. 26-28 illustrate an alternate version of the second mounting portion of the mounting assembly and lockout assembly of the stapling device 10 shown generally as second mounting portion 316 and lockout assembly 312. The second mounting portion 316 includes a distal portion 316a that is received within the proximal portion of the cavity 34 (FIG. 7) defined by the channel 26 of the cartridge assembly 22. In aspects of the disclosure, the second mounting portion 316 defines openings 324, and the anvil 20 and the channel 26 of the cartridge assembly 22 define openings 126, 128 (FIG. 7), respectively. The openings 324, 126, and 128 receive pivot members, e.g., screws or pins 130 (FIG. 7) to pivotably secure the channel 26 to the anvil 20, and to secure the anvil 20 to the second mounting portion 316. The second mounting portion 316 has a substantially planar distal surface 332a, a lower surface 332b (FIG. 28), and transversely extending pivot members 334 (FIG. 26).

The lockout assembly 312 includes a latch 340 and a biasing member 342. The latch 340 defines centrally located openings 344 that receive the pivot members 334 of the second mounting portion 316 to pivotally secure the latch 340 to the second mounting portion 316. The latch 340 is rotatable about the pivot members 334 between a locked position and an unlocked position as described above. The latch 340 includes a distal portion 344a that is positioned distally of the openings 344 and a proximal portion 344b that is positioned proximally of the openings 344. The distal portion 344a of the latch 340 includes a proximally extending finger 346 and a downwardly extending actuator 348. The biasing member 342, e.g., a coil spring, is received about finger 346 and is engaged with the distal surface 332a of the second mounting portion 316 to urge the latch 340 to the locked position. The actuator 348 is positioned within the proximal portion of the channel 26 to engage the fin 59 of the actuation sled 52 of the actuation sled assembly 40 (FIG. 6), as described above regarding actuator 148, to affect movement of the latch 340 from the locked position to the unlocked position.

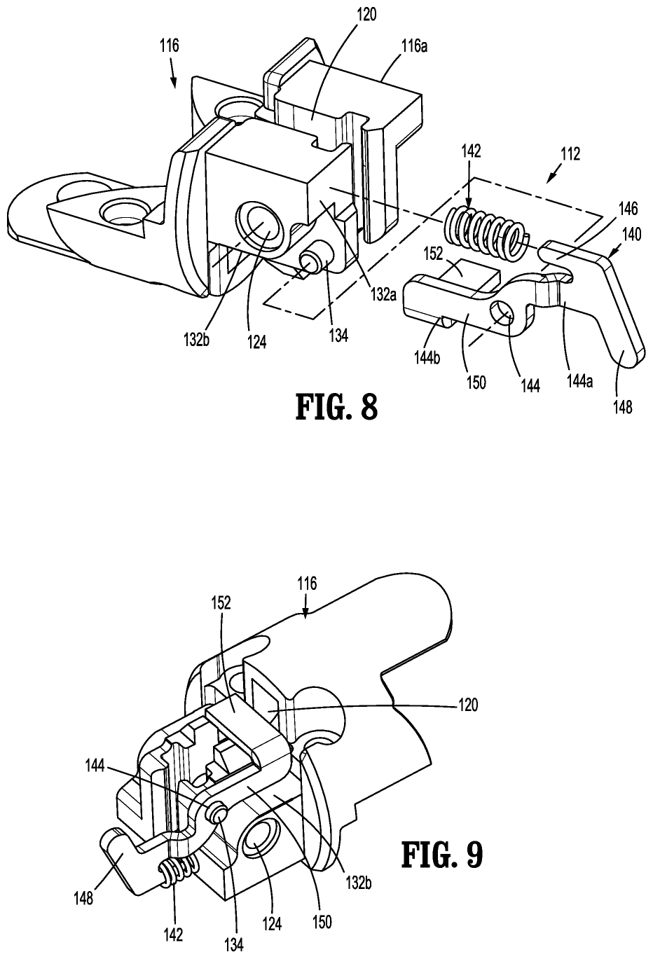

The proximal portion 344b of the latch 340 includes a U-shaped body 350 that includes a transverse stop member 352 and two longitudinally extending legs 354. In aspects of the disclosure, the stop member 352 extends transversely between proximal ends of the legs 354 across the proximal portion of the longitudinal channel 320 of the mounting assembly 110 (FIG. 8) when the latch 340 is in the locked position. The leg 150 is engaged with the lower surface 332b of the second mounting portion 316 when the latch 340 is in the locked position (FIG. 10). Prior to insertion of the staple cartridge 28 into the cavity 34 of the channel 26 (FIG. 7), the latch 340 is positioned in the locked position with the actuator 348 extending into a proximal portion of the cavity 34 of the channel 26. The configuration of the latch 340 provides increased contact surface area on the latch 340 to allow for the load on the latch 340 to be more evenly distributed. Operation of the lockout assembly 312 is identical to that of lockout assembly 112 and will not be described in further detail herein.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A surgical stapling device comprising:
a mounting assembly including a first mounting portion and a second mounting portion secured to the first mounting portion to define a channel;
a tool assembly secured to the mounting assembly and including an anvil and a cartridge assembly, the cartridge assembly including a staple cartridge and a channel, the channel having a proximal portion and a distal portion and defining a cavity, the staple cartridge supported within the cavity of the channel and including a cartridge body, an actuation sled assembly, staples, and pushers, the cartridge body defining a knife slot, and staple receiving pockets positioned on opposite sides of the knife slot, the staples received within the staple receiving pockets, the actuation sled assembly movable through the cartridge body from a sled retracted position to a sled advanced position into engagement with the pushers to eject the staples from the cartridge body, the actuation sled assembly including an actuation sled having an abutment member;
a drive assembly including a flexible drive beam and a working member, the drive assembly movable between a drive retracted position and a drive advanced position to move the actuation sled assembly from the sled retracted position to the sled advanced position, the flexible drive beam extending through the longitudinal channel of the mounting assembly and including a distal portion secured to the working member, the flexible drive beam including a stop surface; and
a lockout assembly having a latch and a biasing member, the latch pivotably secured to the mounting assembly and movable between a locked position and an unlocked position, the latch including a proximal portion and a distal portion, the proximal portion of the latch including a stop member and the distal portion of the latch including a finger and an actuator, the finger supporting the biasing member such that the biasing member is positioned between the mounting assembly and the latch to urge the latch towards the locked position, the actuator positioned to be engaged by the abutment member of the actuation sled as the actuation sled moves from the sled retracted position towards the sled advanced position to move the latch from the locked position to the unlocked position, wherein in the locked position, the stop member of the latch is aligned with the stop surface of the drive assembly.

2. The surgical stapling device of claim 1, wherein the actuation sled of the actuation sled assembly includes a central body portion, and the actuation sled assembly includes a knife that is supported on the central body portion of the actuation sled, the knife positioned to engage the working member of the drive assembly as the drive assembly moves from the drive retracted position towards the drive advanced position to move the actuation sled assembly from the sled retracted position to the sled advanced position.

3. The surgical stapling device of claim 2, wherein the knife is positioned proximally of the central body portion of the actuation sled.

4. The surgical stapling device of claim 3, wherein a distance between the working member of the drive assembly and the central body portion of the actuation sled when the drive assembly is in the drive retracted position and the actuation sled assembly is in the sled retracted position is greater than a distance between the stop surface of the drive assembly and the stop member of the latch of the lockout assembly.

5. The surgical stapling device of claim 2, wherein the knife is supported on the actuation sled for movement between a lowered position and a raised position.

6. The surgical stapling device of claim 5, wherein the working member of the drive assembly includes a first beam, a second beam, and a vertical strut connecting the first beam to the second beam, the vertical strut defining a cam slot, and the knife includes a cam member that is received in the cam slot when the drive assembly is moved from the drive retracted position towards the drive advanced position to move the knife from the lowered position to the raised position.

7. The surgical stapling device of claim 1, wherein the anvil is fixedly secured to the mounting assembly, and the cartridge assembly is pivotably secured to the mounting assembly and movable in relation to the anvil between the unclamped position and the clamped position.

8. The surgical stapling device of claim 1, wherein the latch includes a central portion that includes at least one opening and the mounting assembly includes at least one pivot member that is received within the at least one opening to pivotably couple the latch to the mounting assembly.

9. The surgical stapling device of claim 8, wherein the proximal portion of the latch is U-shaped and includes two spaced longitudinally extending legs, the stop member extending transversely between proximal portions of the longitudinally extending legs.

10. The surgical stapling device of claim 8, wherein the proximal portion of the latch is L-shaped and includes a longitudinally extending leg, the stop member extending transversely from a proximal portion of the longitudinally extending leg.

11. The surgical stapling device of claim 4, wherein the actuation sled includes a base having a cutout positioned proximally of the central body portion of the actuation sled, the cutout positioned to receive the working member of the drive assembly when the drive assembly is moved from the drive retracted position towards the drive advanced position.

12. The surgical stapling device of claim 1, wherein the working member has an I-beam configuration and includes a first beam, a second beam, and a vertical strut that interconnects the first beam and the second beam, the vertical strut including a distal surface that engages the knife of the actuation sled assembly when the drive assembly is moved from the drive retracted position towards the drive advanced position.

13. The surgical stapling device of claim 12, wherein the distal surface of the vertical strut of the drive assembly defines a recess that receives the knife when the drive assembly is moved from the drive retracted position towards the drive advanced position.

14. A surgical stapling device comprising:
a mounting assembly including a first mounting portion and a second mounting portion secured to the first mounting portion to define a channel;
a tool assembly secured to the mounting assembly and including an anvil and a cartridge assembly, the cartridge assembly including a staple cartridge and a channel, the channel having a proximal portion and a distal portion and defining a cavity, the staple cartridge supported within the cavity of the channel and including a cartridge body, an actuation sled assembly, staples, and pushers, the cartridge body defining a knife slot and staple receiving pockets positioned on opposite sides of the knife slot, the staples received within the staple receiving pockets of the cartridge body, the actuation sled assembly movable through the cartridge body from a sled retracted position to a sled advanced position into engagement with the pushers to eject the staples from the cartridge body, the actuation sled assembly including an actuation sled having a central body portion and a knife supported on the central body portion, the knife extending proximally of the central body portion of the actuation sled;
a drive assembly including a stop surface, the drive assembly positioned to engage the knife of the actuation sled assembly as the drive assembly moves from the drive retracted position towards the drive advanced position to move the actuation sled assembly from the sled retracted position to the sled advanced position; and
a lockout assembly having a latch pivotably secured to the mounting assembly and movable between a locked position and an unlocked position, the latch including a stop member that is aligned with the stop surface of the drive assembly in the locked position;
wherein a distance between the drive assembly and the central body portion of the actuation sled when the drive assembly is in the drive retracted position and the actuation sled assembly is in the sled retracted position is greater than a distance between the stop surface of the drive assembly and the stop member of the latch of the lockout assembly such that the latch will prohibit movement of the drive assembly to the drive advanced position when a knife is not present in the staple cartridge.

15. The surgical stapling device of claim 14, wherein the knife is supported on the actuation sled for movement between a lowered position and a raised position.

16. The surgical stapling device of claim 15, wherein the working member of the drive assembly includes a first beam, a second beam, and a vertical strut connecting the first beam to the second beam, the vertical strut defining a cam slot, and the knife includes a cam member that is received in the cam slot when the drive assembly is moved from the drive retracted position towards the drive advanced position to move the knife from the lowered position to the raised position.

17. The surgical stapling device of claim 14, wherein the lockout assembly includes a biasing member to urge the latch towards the locked position.

18. The surgical stapling device of claim 17, wherein the latch includes a finger and an actuator, the finger supporting the biasing member to urge the latch towards the locked position, and the actuator positioned to be engaged by the abutment member of the actuation sled as the actuation sled moves from the sled retracted position towards the sled advanced position to move the latch from the locked position to the unlocked position.

19. The surgical stapling device of claim 18, wherein latch includes a proximal portion having a U-shape and including two spaced longitudinally extending legs, the stop member extending transversely between proximal portions of the longitudinally extending legs.

20. A surgical stapling device comprising:
a mounting assembly;
a tool assembly secured to the mounting assembly and including an anvil and a cartridge assembly, the cartridge assembly including a staple cartridge and a channel, the channel having a proximal portion and a distal portion and defining a cavity, the staple cartridge supported within the cavity of the channel and including a cartridge body, an actuation sled, staples, and pushers, the cartridge body defining a knife slot, and staple receiving pockets positioned on opposite sides of the knife slot, and the staples received within the staple receiving pockets, the actuation sled movable through the cartridge body from a sled retracted position to a sled advanced position into engagement with the pushers to eject the staples from the cartridge body;
a drive assembly movable between a drive retracted position and a drive advanced position to move the actuation sled assembly from the sled retracted position to the sled advanced position, the drive assembly including a stop surface; and
a lockout assembly having a latch pivotably secured to the mounting assembly and movable between a locked position and an unlocked position, the latch including a proximal portion and a distal portion, the proximal portion of the latch including a stop member and the distal portion of the latch including an actuator, the actuator positioned to be engaged by the abutment member on the actuation sled as the actuation sled moves from the sled retracted position towards the sled advanced position to move the latch from the locked position to the unlocked position, wherein in the locked position, the stop member of the latch is aligned with the stop surface of the drive assembly.
